(19) United States
(12) Patent Application Publication
(10) Pub. No.: US 2020/0245634 A1
(43) Pub. Date: Aug. 6, 2020

(54) COMPOSITION AND METHOD FOR SUPPRESSING DIGESTIVE TRACT SYMPTOMS

(71) Applicant: MEIJI CO., LTD., Chuo-ku, Tokyo (JP)

(72) Inventors: Kentaro NAKAMURA, Tokyo (JP); Kinya ASHIDA, Tokyo (JP); Tadahito MAEKAWA, Tokyo (JP); Noriyoshi ARAKI, Tokyo (JP)

(21) Appl. No.: 16/621,027

(22) PCT Filed: Jun. 11, 2018
(86) PCT No.: PCT/JP2018/022177

§ 371 Date: Dec. 10, 2019

(30) Foreign Application Priority Data
Jun. 12, 2017 (JP) 2017-115174

(51) Int. Cl. A23D 9/00; A23L 33/115; A23L 5/00; A61K 31/23

(52) U.S. Cl. CPC A23D 9/00; A61K 31/23; A23L 5/00; A23L 33/115

(57) ABSTRACT

A composition for suppressing digestive tract symptoms arising from ingestion of a medium chain fatty acid-containing lipid, including a medium chain fatty acid-containing lipid and a long chain fatty acid-containing lipid.

### TECHNICAL FIELD

The present invention relates to a composition and a method for suppressing digestive tract symptoms. More specifically, the present invention relates to a composition and a method for suppressing digestive tract symptoms arising from ingestion of a medium chain fatty acid-containing lipid.

### BACKGROUND ART

A ketone body is a generic term for acetoacetic acid, β-hydroxybutyric acid and acetone, and is synthesized by β-oxidation of fatty acid in a liver in vivo. The ketone body produced is utilized as energy similarly to glucose or the like in many tissues other than the liver, such as a brain, a heart and a skeletal muscle. In particular, in the brain, only the glucose and the ketone body are utilized, whereby the ketone body serves as an important energy source (Non-Patent Document 1).

The fatty acid serving as a main raw material for producing the ketone body in vivo is classified into long chain fatty acid, medium chain fatty acid and short chain fatty acid according to a length of a carbon chain. It is considered that the medium chain fatty acid can produce the ketone body more efficiently than the long chain fatty acid by differences in their absorption and metabolic pathways. Moreover, it is known that a blood ketone body concentration further rises in humans or animals by ingestion of a medium chain fatty acid-containing lipid in comparison with ingestion of a long chain fatty acid-containing lipid (Non-Patent Document 2).

However, it is known that uncomfortable symptoms may be felt in a digestive tract including a stomach when the medium chain fatty acid-containing lipid is ingested.

### RELATED ART DOCUMENTS

### Non-Patent Documents

Non-Patent Document 1: Cahill G F Jr., “Fuel metabolism in starvation.,” Annual Review of Nutrition, Aug. 21, 2006, vol. 21, pp. 1-22
Non-Patent Document 2: F X Pi-Sunyer, and 2 others, “Insulin and ketone responses to ingestion of medium and long-chain triglycerides in man.,” Diabetes, Feb. 1969, 18(2), pp. 96-100

### SUMMARY OF THE INVENTION

An objective of the present invention is to provide a composition and a method for suppressing uncomfortable symptoms in a digestive tract including a stomach when a medium chain fatty acid-containing lipid is ingested.

The present inventors have diligently conducted research in view of the above-described problem, and as a result, they have found that, when a human ingests a composition obtained by blending a long chain fatty acid-containing lipid with a medium chain fatty acid-containing lipid, digestive tract symptoms when the medium chain fatty acid-containing lipid is ingested can be suppressed.

Accordingly, the present invention can provide the composition for suppressing the digestive tract symptoms, and the like, as described below.

1. A composition for suppressing digestive tract symptoms arising from ingestion of a medium chain fatty acid-containing lipid, comprising the medium chain fatty acid-containing lipid and a long chain fatty acid-containing lipid.

2. The composition according to 1, wherein the digestive tract symptoms are upper digestive tract symptoms.

3. The composition according to 1 or 2, wherein the digestive tract symptoms are symptoms in at least one of an esophagus, a stomach and a duodenum.

4. The composition according to any one of 1 to 3, wherein the medium chain fatty acid-containing lipid contains a constituent fatty acid having 6 to 12 carbon atoms, and the long chain fatty acid-containing lipid contains a constituent fatty acid having 13 to 30 carbon atoms.

5. The composition according to any one of 1 to 4, wherein the medium chain fatty acid-containing lipid contains octanoic acid and decanoic acid as the constituent fatty acid, and the long chain fatty acid-containing lipid contains palmitic acid, oleic acid and linoleic acid as the constituent fatty acid.

6. The composition according to any one of 1 to 5, wherein a blending amount of the medium chain fatty acid-containing lipid is 3 g or more and 15 g or less per 100 g of the composition, and a blending proportion of the long chain fatty acid-containing lipid is 30 mass % or more and 200 mass % or less based on the blending amount of the medium chain fatty acid-containing lipid.

7. The composition according to any one of 1 to 6, wherein the composition is in a form of a fluid diet, a semi-fluid diet, jelly, gel, powder, formulated milk powder, fermented milk, a bar, mousse, chocolate, a biscuit or ice cream.

8. A method for suppressing digestive tract symptoms arising from ingestion of a medium chain fatty acid-containing lipid, comprising ingesting a composition obtained by blending a long chain fatty acid-containing lipid with the medium chain fatty acid-containing lipid.

9. The method according to 8, wherein the digestive tract symptoms are upper digestive tract symptoms.

10. The method according to 8 or 9, wherein the digestive tract symptoms are symptoms in at least one of an esophagus, a stomach and a duodenum.

11. The method according to any one of 8 to 10, wherein the medium chain fatty acid-containing lipid contains a constituent fatty acid having 6 to 12 carbon atoms, and the long chain fatty acid-containing lipid contains a constituent fatty acid having 13 to 30 carbon atoms.

12. The method according to any one of 8 to 11, wherein the medium chain fatty acid-containing lipid contains octanoic acid and decanoic acid as a constituent fatty acid, and the long chain fatty acid-containing lipid contains palmitic acid, oleic acid and linoleic acid as a constituent fatty acid.

13. The method according to any one of 8 to 12, wherein a blending amount of the medium chain fatty acid-containing lipid is 3 g or more and 15 g or less per 100 g of the composition, and a blending proportion of the long chain fatty acid-containing lipid is 30 mass % or more and 200 mass % or less based on the blending amount of the medium chain fatty acid-containing lipid.

14. The method according to any one of 8 to 13, wherein the composition is in a form of a fluid diet, a semi-fluid diet, jelly, gel, powder, formulated milk powder, fermented milk, a bar, mousse, chocolate, a biscuit or ice cream.

15. Use of a composition comprising a medium chain fatty acid-containing lipid and a long chain fatty acid-containing lipid, for suppressing digestive tract symptoms arising from ingestion of the medium chain fatty acid-containing lipid.

16. The use according to 15, wherein the digestive tract symptoms are upper digestive tract symptoms.

17. The use according to 15 or 16, wherein the digestive tract symptoms are symptoms in at least one of an esophagus, a stomach and a duodenum.

18. The use according to any one of 15 to 17, wherein the medium chain fatty acid-containing lipid contains a constituent fatty acid having 6 to 12 carbon atoms, and the long chain fatty acid-containing lipid contains a constituent fatty acid having 13 to 30 carbon atoms.

19. The use according to any one of 15 to 18, wherein the medium chain fatty acid-containing lipid contains octanoic acid and decanoic acid as a constituent fatty acid, and the long chain fatty acid-containing lipid contains palmitic acid, oleic acid and linoleic acid as a constituent fatty acid.

20. The use according to any one of 15 to 19, wherein a blending amount of the medium chain fatty acid-containing lipid is 3 g or more and 15 g or less per 100 g of the composition, and a blending proportion of the long chain fatty acid-containing lipid is 30 mass % or more and 200 mass % or less based on the blending amount of the medium chain fatty acid-containing lipid.

21. The use according to any one of 15 to 20, wherein the composition is in a form of a fluid diet, a semi-fluid diet, jelly, gel, powder, formulated milk powder, fermented milk, a bar, mousse, chocolate, a biscuit or ice cream.

22. Use of a composition comprising a medium chain fatty acid-containing lipid and a long chain fatty add-containing lipid, for producing a composition for suppressing digestive tract symptoms arising from ingestion of the medium chain fatty acid-containing lipid.

23. The use according to 22, wherein the digestive tract symptoms are upper digestive tract symptoms.

24. The use according to 22 or 23, wherein the digestive tract symptoms are symptoms in at least one of an esophagus, a stomach and a duodenum.

25. The use according to any one of 22 to 24, wherein the medium chain fatty acid-containing lipid contains a constituent fatty acid having 6 to 12 carbon atoms, and the long chain fatty acid-containing lipid contains a constituent fatty acid having 13 to 30 carbon atoms.

26. The use according to any one of 22 to 25, wherein the medium chain fatty acid-containing lipid contains octanoic acid and decanoic acid as a constituent fatty acid, and the long chain fatty acid-containing lipid contains palmitic acid, oleic acid and linoleic acid as a constituent fatty acid.

27. The use according to any one of 22 to 26, wherein a blending amount of the medium chain fatty acid-containing lipid is 3 g or more and 15 g or less per 100 g of the composition, and a blending proportion of the long chain fatty acid-containing lipid is 30 mass % or more and 200 mass % or less based on the blending amount of the medium chain fatty acid-containing lipid.

The present invention can provide a composition and a method for suppressing uncomfortable symptoms in a digestive tract including a stomach when a medium chain fatty acid-containing lipid is ingested.

### BRIEF DESCRIPTION OF THE DRAWINGS

FIG. 1 shows a maximum blood concentration (Cmax) of β-hydroxybutyric acid by 3 hours after ingestion of each test food.

FIG. 2 shows an area under the blood concentration-time curve (AUC) of β-hydroxybutyric acid by 3 hours after ingestion of each test food.

### DESCRIPTION OF THE EMBODIMENTS

### [Composition for Suppressing Digestive Tract Symptoms]

The composition of the present invention is a composition for suppressing digestive tract symptoms arising from ingestion of a medium chain fatty acid-containing lipid, the composition comprising a medium chain fatty acid-containing lipid and a long chain fatty acid-containing lipid.

The composition of the present invention can suppress various symptoms of a digestive tract (an esophagus, a stomach or an intestine), arising from ingestion of the medium chain fatty acid-containing lipid. Specific examples of the digestive tract symptoms include, but not be limited to, irritation, an ache, a hot feeling, churning of stomach, bloating, burping, nausea, discomfort, an uncomfortable feeling, and the like. The composition of the present invention can suppress these symptoms in the digestive tract.

In one embodiment of the present invention, the term “digestive tract” means particularly an upper digestive tract (the esophagus, the stomach, or a duodenum). Thus, according to one embodiment of the present invention, various symptoms in at least one of the esophagus, the stomach and the duodenum, arising from ingestion of the medium chain fatty acid-containing lipid, can be suppressed.

As a diet devised so that a great number of ketone bodies may be produced in vivo, a high-fat and low-carbohydrate diet (ketogenic diet) has been known for a long time. A blood ketone body concentration rises by ingestion of the ketogenic diet. It is known that the ketogenic diet is effective in treatment of GLUT1 deficiency or suppressing seizures in intractable epilepsy (Tatsuya Fujii, “Fundamental to Practice of Ketogenic Diet (“Ketonshoku no kiso kara jissen made” in Japanese),” SHINDAN TO CHIRYO SHA, Inc., March, 2011). Further, it has recently been reported that the ketogenic diet is also effective in prevention and treatment of neurodegenerative disease including the Alzheimer's disease and the like (Maciej Gasior, and 2 others, “Neuroprotective and disease-modifying effects of the ketogenic diet,” Behav Pharmacol., Sep. 2006, 17(5-6), pp. 431-439). Accordingly, upon ingesting a large amount of the medium chain fatty acid-containing lipids in order to prevent the above-described diseases or relieve the symptoms, the composition of the present invention, and the method of the present invention described later can suppress the digestive tract symptoms to improve QOL.

In the present invention, the term “medium chain fatty acid-containing lipid” means a lipid in which the constituent fatty acid therein has a medium chain in length, and is also referred to as Medium Chain Triglyceride (MCT). The term “MCT” means typically a lipid formed of fatty acid having 6 to 12 carbon atoms, preferably a lipid formed of fatty acid having 8 to 12 carbon atoms, and more preferably a lipid formed of fatty acid having 8 to 10 carbon atoms. Specific examples of the medium chain fatty acid include hexanoic acid, heptanoic acid, octanoic acid, nonanoic acid, decanoic acid and dodecanoic acid. Three medium chain fatty acids bonded to one glycerol, forming the lipid, may be identical to or different from each other. When a plurality of medium chain fatty acids are bonded thereto, proportions thereof are not particularly limited.

In one embodiment of the present invention, the medium chain fatty acid-containing lipid is a lipid containing at least one of octanoic acid and decanoic acid, for example, and preferably a lipid containing octanoic acid and decanoic acid.

Moreover, in one embodiment of the present invention, the medium chain fatty acid-containing lipid is preferably a lipid containing octanoic acid and decanoic acid in a mass ratio in the range of 50 to 100:50 to 0, more preferably a lipid containing octanoic acid and decanoic acid in a mass ratio in the range of 70 to 90:30 to 10, and particularly preferably a lipid containing octanoic acid and decanoic acid in a mass ratio of 75:25.

In the present invention, the term “long chain fatty acid-containing lipid” means a lipid in which the constituent fatty acid therein has a long chain in length, and is also referred to as Long Chain Triglyceride (LCT). The term “LCT” means typically a lipid formed of fatty acid having 13 to 30 carbon atoms, preferably a lipid formed of fatty acid having 13 to 24 carbon atoms, and more preferably a lipid formed of fatty acid having 13 to 18 carbon atoms. Specific examples of the long chain fatty acid include tetradecanoic acid, pentadecanoic acid, hexadecanoic acid, 9-hexadecenoic acid, octadecanoic acid, cis-9-octadecenoic acid, 11-octadecenoic acid, tetracosanoic acid, cis-15-tetracosanoic acid and triacontanoic acid, and the long chain fatty acid may be unsaturated fatty acid. Three long chain fatty acids bonded to one glycerol, forming the lipid, may be identical to or different from each other. When a plurality of long chain fatty acids are bonded thereto, proportions thereof are not particularly limited.

In one embodiment of the present invention, the long chain fatty add-containing lipid is preferably a lipid containing palmitic acid, oleic acid and linoleic acid, and more preferably a lipid containing palmitic acid, oleic acid and linoleic add in a mass ratio in the range of 20 to 30:50 to 60:10 to 20.

The medium chain fatty acid-containing lipid exists in seeds of plant bodies such as coconut and palm fruit, or milk, dairy products or the like, and therefore the lipid extracted (including a crude extract) or purified (including a crude product) therefrom can be used in the present invention. The long chain fatty acid-containing lipid exists in seeds of plant bodies such as soybean, rapeseed and olive, or beef tallow or lard, and therefore the lipid extracted (including a crude extract) or purified (including a crude product) therefrom can be used in the present invention.

In the present invention, the term “composition” means a material to be orally ingested or ingested through a tube (a gastric fistula, an intestinal fistula, or a nasogastric tube). A form of the composition is not particularly limited, and examples thereof include solid, semi-solid material, liquid, fluid and paste. Accordingly, the composition of the present invention can be orally ingested or ingested through the tube (the gastric fistula, the intestinal fistula, or the nasogastric tube) as a diet, a beverage, confectionery, a nutrient, a supplement or a pharmaceutical, for example. Specifically, the composition of the present invention can be produced in the form such as a fluid diet, a semi-fluid diet, jelly, gel, powder, formulated milk powder, fermented milk, a bar, mousse, chocolate, a biscuit or ice cream.

In the present invention, a blending proportion of the medium chain fatty acid-containing lipid in the composition is not particularly limited, and can be appropriately determined depending on an application or a function of the composition. For example, the lipid is contained preferably in 3 g or more and 15 g or less, more preferably in 5 g or more and 12 g or less, and further preferably in 7 g or more and 10 g or less, per 100 g of the composition.

In the present invention, a blending proportion of the long chain fatty acid-containing lipid in the composition is preferably 30 mass % or more and 200 mass % or less, more preferably 50 mass % or more and 120 mass % or less, and further preferably 70 mass % or more and 100 mass % or less, based on a blending amount of MCT.

In the present invention, the composition can contain an arbitrary component in addition to the medium chain fatty acid-containing lipid and the long chain fatty add-containing lipid. Specific examples of the arbitrary component include water, a protein, a lipid, a glucide, dietary fiber, vitamins, minerals, amino acid, a sweetener, flavors, an antioxidant, an emulsifier, a thickening agent, a stabilizing agent, a gelling agent and a pH adjuster.

When the composition of the present invention is ingested, no significant difference is found in the blood ketone body concentration (particularly, a β-hydroxybutyric acid concentration), as compared with the case where a lipid containing only medium chain fatty acid is ingested, and the blood ketone body concentration (particularly, the β-hydroxybutyric acid concentration) can be raised in a similar manner.

### [Method for Suppressing Digestive Tract Symptoms]

The method of the present invention is a method for suppressing the digestive tract symptoms arising from ingestion of the medium chain fatty acid-containing lipid, the method being characterized by ingesting the composition containing the medium chain fatty acid-containing lipid and the long chain fatty acid-containing lipid.

The method of the present invention can suppress various symptoms (for example, irritation, the ache, the hot feeling, churning of stomach, bloating, burping, nausea, discomfort, the uncomfortable feeling, and the like) of the digestive tract (the esophagus, the stomach or the intestine), arising from ingestion of the medium chain fatty acid-containing lipid.

In one embodiment of the present invention, the term “digestive tract” means particularly the upper digestive tract (the esophagus, the stomach or the duodenum). Thus, according to one embodiment of the present invention, various symptoms in at least one of the esophagus, the stomach and the duodenum, arising from ingestion of the medium chain fatty acid-containing lipid, can be suppressed.

In the method of the present invention, the term “composition containing the medium chain fatty acid-containing lipid and the long chain fatty acid-containing lipid means the composition of the present invention already described. The specific features about the composition of the present invention is as already described.

Hereinafter, the present invention will be further specifically described by providing Examples, but the scope of the present invention is not limited to the description in the Examples.

### EXAMPLES

### Test Example 1: Evaluation of Upper Digestive Tract Symptoms

Two test foods obtained by blending medium chain fatty acid and long chain fatty acid, and 2 test foods serving as a control obtained by blending medium chain fatty acid were prepared, the test foods were ingested by 15 subjects (13 men, 2 women, 26 to 57 years of age) according to a cross-over trial method, and development of a bodily feeling of upper digestive tract symptoms after ingestion was compared.

In the cross-over trial method, each subject ingested all 4 test foods, but the order of ingesting the 4 test foods was randomized, and is different depending on the subject.

Table 1 shows component compositions of the 4 test foods prepared.

TABLE 1

| | | Test | Test | Test | Test |
|---|---|---|---|---|---|
| | | food 1 | food 2 | food 3 | food 4 |
| Form | | Fluid diet | Fluid diet | Jelly | Jelly |
| Capacity | | 125 g | 125 g | 128 g | 128 g |
| Protein (g) | | 3.8 | 3.8 | 3.8 | 3.8 |
| Protein | Casein sodium | 2.9 | 2.9 | 1.8 | 1.8 |
| contents | salt (g) | | | | |
| | Whey protein | 0.9 | 0.9 | 0.6 | 0.6 |
| | isolate (g) | | | | |
| | Gelatin (g) | — | — | 1.4 | 1.4 |
| Lipid (g) | | 10 | 18.1 | 10 | 18.1 |
| Lipid | MCT (g) | 10 | 10 | 10 | 10 |
| contents | LCT (g) | — | 8.1 | — | 8.1 |

| | | | | |
|---|---|---|---|---|
| Carbohydrate (g) | 3.3 | 3.3 | 3.3 | 3.3 |
| Dietary fiber | 2.5 | 2.5 | 2.5 | 2.5 |
| (indigestible dextrin) (g), | | | | |
| among the carbohydrate | | | | |
| Calorie (kcal) | 111 | 184 | 111 | 184 |

Of the lipids, a medium chain fatty acid-containing lipid (MCT) is a lipid containing octanoic acid (number of carbon atoms: 8) and decanoic acid (number of carbon atoms: 10) in a mass ratio of 75:25, and a long chain fatty acid-containing lipid (LCT) is a lipid containing palmitic acid (number of carbon atoms: 16), oleic acid (number of carbon atoms: 18), linoleic acid (number of carbon atoms: 18) and other fatty acids in a mass ratio of 21:52:16:11.

In each test day, the subjects were fasted for 12 hours before start of the test. The subjects were evaluated in a bodily feeling of upper digestive tract symptoms in 13 points in total, namely, before ingestion of the test foods and every 15 minutes from the ingestion until 3 hours after the ingestion (before the ingestion, 15 minutes after the ingestion, 30 minutes thereafter, 45 minutes thereafter, 60 minutes thereafter, 75 minutes thereafter, 90 minutes thereafter, 105 minutes thereafter, 120 minutes thereafter, 135 minutes thereafter, 150 minutes thereafter, 165 minutes thereafter and 180 minutes thereafter).

Evaluation items on the upper digestive tract symptoms are 6 in the number, including “irritated in an upper abdomen (stomachache),” “felt hot in the upper abdomen,” “churned in a stomach (upset stomach),” “felt bloating in an abdomen,” “burped,” and “felt nauseous,” and the bodily feeling was scored in each evaluation item on a 3 rating scale from 0 to 2 (“not applicable”: 0, “slightly applicable”: 1, and “applicable”: 2).

Table 2 shows the results of the bodily feeling score. In Table 2, a numeric value of each evaluation item was obtained by calculating a total of the bodily feeling score in 13 points for every subject, and calculating a value: average value±standard deviation among all the subjects. Accordingly, an upper limit of the bodily feeling score of each evaluation item is 26 (2×13 points), and an upper limit of a total score of the 6 evaluation items on the upper digestive tract symptoms is 156 (26×6 evaluation items).

TABLE 2

| Evaluation items on upper | | | | |
|---|---|---|---|---|
| digestive tract symptoms | Test food 1 | Test food 2 | Test food 3 | Test food 4 |
| Irritated in the upper | 3.5 ± 3.4 | 1.7 ± 2.6 | 1.9 ± 2.6 | 1.9 ± 2.1 |
| abdomen (stomachache) | | | | |
| Felt hot in the | 3.5 ± 3.9 | 2.2 ± 2.7 | 2.7 ± 2.8 | 1.7 ± 2.6 |
| upper abdomen | | | | |
| Churned in the stomach | 4.3 ± 4.6 | 2.4 ± 2.9 | 2.6 ± 3.3 | 2.9 ± 4.3 |
| (upset stomach) | | | | |
| Felt bloating in | 0.5 ± 1.0 | 0.0 ± 0.0 | 1.1 ± 1.6 | 0.5 ± 1.4 |
| the abdomen | | | | |
| Burped | 1.1 ± 1.3 | 0.6 ± 0.9 | 0.9 ± 1.5 | 1.2 ± 1.9 |
| Felt nauseous | 0.2 ± 0.6 | 0.1 ± 0.3 | 0.0 ± 0.0 | 0.0 ± 0.0 |
| Total score of upper | 13.1 ± 11.8 | 6.9 ± 6.0 | 9.3 ± 6.4 | 8.3 ± 9.1 |
| digestive tract symptoms | | | | |

In a total score of the upper digestive tract symptoms when a fluid diet-form test food was ingested, a statistically significant difference was found in the test food 2 obtained by blending the long chain fatty acid, as compared with the test food 1 as the control without blending the long chain fatty acid, and the upper digestive tract symptoms were significantly suppressed.

Moreover, in a total score of the upper digestive tract symptoms when a jelly-form test food was ingested, a difference was found in the test food 4 obtained by blending long chain fatty acid, as compared with the test food 3 as the control without blending the long chain fatty acid, and the upper digestive tract symptoms were suppressed.

### Test Example 2 Evaluation of a Blood Ketone Body Concentration

Two test foods obtained by blending medium chain fatty acid and long chain fatty acid and 2 test foods serving as a control obtained by blending medium chain fatty acid were prepared, the test foods were ingested by 2 subjects (2 men, 37 and 48 years of age), and a change in a blood β-hydroxybutyric acid concentration after the ingestion was observed. The blood β-hydroxybutyric acid concentration was measured with Bundle Kit (Precision, Abbott).

Component compositions of the 4 test foods prepared are the same as in Table 1.

The subjects were fasted for 12 hours before start of a test. The subjects were measured in the blood β-hydroxybutyric acid concentration in 7 points in total, namely, before ingestion of the test foods and every 30 minutes from the ingestion until 3 hours after the ingestion (before the ingestion, 30 minutes after the ingestion, 60 minutes thereafter, 90 minutes thereafter, 120 minutes thereafter, 150 minutes thereafter and 180 minutes thereafter).

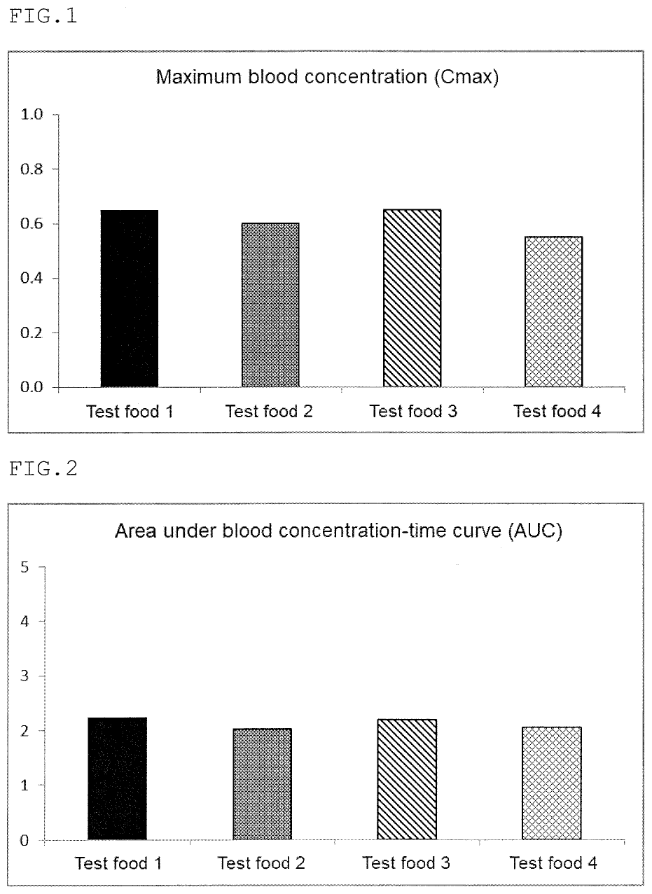

FIG. 1 and FIG. 2 show the results of a maximum blood concentration (Cmax: unit “mmol/L”) of β-hydroxybutyric acid and an area under the blood concentration-time curve (AUC: unit “mol·hour/0.5·L”), respectively. Both FIG. 1 and FIG. 2 show average values of measured values in 2 subjects. No significant difference was found in Cmax and AUC even when all test foods were ingested.

This finding indicates that, when the composition of the present invention is ingested, the digestive tract symptoms can be suppressed, and simultaneously no significant difference is found in a concentration of β-hydroxybutyric acid, which is a ketone body in blood, as compared with the case where only MCT is ingested, and the concentration of β-hydroxybutyric acid, which is the ketone body in blood, can be raised.

### INDUSTRIAL APPLICABILITY

The present invention can suppress various symptoms of a digestive tract (an esophagus, a stomach, or an intestine), arising from ingestion of a medium chain fatty acid-containing lipid. Moreover, upon ingesting a large amount of the medium chain fatty acid-containing lipids in order to prevent specific diseases or relieve the symptoms, the present invention can suppress digestive tract symptoms to improve QOL.

Several embodiments and/or Examples of the present invention have been described in detail above, but those skilled in the art will readily make a great number of modifications to the exemplary embodiments and/or Examples without substantially departing from new teachings and advantageous effects of the present invention. Accordingly, all such modifications are included within the scope of the present invention.

The entire contents of the documents described in the description concerning the present application and the description of the Japanese application serving as a basis of claiming the priority concerning the present application to the Paris Convention are incorporated by reference herein.

1.-7. (canceled)

8. A method for suppressing digestive tract symptoms arising from ingestion of a medium chain fatty acid-containing lipid, comprising ingesting a composition obtained by blending a long chain fatty acid-containing lipid with the medium chain fatty acid-containing lipid.

9. (canceled)

10. The method according to claim 8, wherein the digestive tract symptoms are upper digestive tract symptoms.

11. The method according to claim 8, wherein the digestive tract symptoms are symptoms in at least one of an esophagus, a stomach and a duodenum.

12. The method according to claim 8, wherein the medium chain fatty acid-containing lipid contains a constituent fatty acid having 6 to 12 carbon atoms, and the long chain fatty acid-containing lipid contains a constituent fatty acid having 13 to 30 carbon atom.

13. The method according to claim 8, wherein the medium chain fatty acid-containing lipid contains octanoic acid and decanoic acid as a constituent fatty acid, and the long chain fatty acid-containing lipid contains palmitic acid, oleic acid and linoleic acid as a constituent fatty acid.

14. The method according to claim 8, wherein a blending amount of the medium chain fatty acid-containing lipid is 3 g or more and 15 g or less per 100 g of the composition, and a blending proportion of the long chain fatty acid-containing lipid is 30 mass % or more and 200 mass % or less based on the blending amount of the medium chain fatty acid-containing lipid.

15. The method according to claim 8, wherein the composition is in a form of a fluid diet, a semi-fluid diet, jelly, gel, powder, formulated milk powder, fermented milk, a bar, mousse, chocolate, a biscuit or ice cream.

16. A composition for suppressing digestive tract symptoms arising from ingestion of a medium chain fatty acid-containing lipid, comprising the medium chain fatty acid-containing lipid and a long chain fatty acid-containing lipid.

17. The composition according to claim 16, wherein the digestive tract symptoms are upper digestive tract symptoms.

18. The composition according to claim 16, wherein the digestive tract symptoms are symptoms in at least one of an esophagus, a stomach and a duodenum.

19. The composition according to claim 16, wherein the medium chain fatty acid-containing lipid contains a constituent fatty acid having 6 to 12 carbon atoms, and the long chain fatty acid-containing lipid contains a constituent fatty acid having 13 to 30 carbon atoms.

20. The composition according to claim 16, wherein the medium chain fatty acid-containing lipid contains octanoic acid and decanoic acid as a constituent fatty acid, and the long chain fatty acid-containing lipid contains palmitic acid, oleic acid and linoleic acid as a constituent fatty acid.

21. The composition according to claim 16, wherein a blending amount of the medium chain fatty acid-containing lipid is 3 g or more and 15 g or less per 100 g of the composition, and a blending proportion of the long chain fatty acid-containing lipid is 30 mass % or more and 200 mass % or less based on the blending amount of the medium chain fatty acid-containing lipid.

22. The composition according to claim 16, wherein the composition is in a form of a fluid diet, a semi-fluid diet, jelly, gel, powder, formulated milk powder, fermented milk, a bar, mousse, chocolate, a biscuit or ice cream.
